(19) United States
(12) Patent Application Publication
(10) Pub. No.: US 2008/0194823 A1
(43) Pub. Date: Aug. 14, 2008

(54) PREPARATION OF LORATADINE FORM I

(76) Inventors: Mayur Devjibhai Khunt, Junagadh (IN); Naveen Kumar Reddy Keshava, Hyderabad (IN); Srinivasa Reddy Madduri, Guntur (IN); Somaiah S. Sripathi, Nalgonda (IN); Sudhakar Valluri, West Godavari (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/696,391

(22) Filed: Apr. 4, 2007

Related U.S. Application Data
(60) Provisional application No. 60/820,185, filed on Jul. 24, 2006.

(30) Foreign Application Priority Data
Apr. 4, 2006 (IN) 613/CHE/2006

(51) Int. Cl. C07D 221/16

(52) U.S. Cl. 546/93

(57) ABSTRACT

A process for preparing loratadine crystalline polymorphic Form I.

### INTRODUCTION TO THE INVENTION

The present invention relates to a process for the preparation of crystalline polymorphic Form I of loratadine. In an aspect, the present invention relates to a process for the preparation of polymorphic Form I of loratadine substantially free of polymorphic Form II.

Loratadine has a chemical name ethyl 4-(8-chloro-5,6-dihydro-11H-benzo[5,6]cyclohepta[1,2-b]pyridin-11-ylidene)-1-piperidinecarboxylate, and is structurally represented by Formula I.

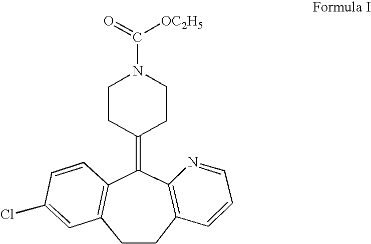

Loratadine is a long-acting tricyclic antihistamine with selective peripheral histamine H₁-receptor antagonistic activity. Pharmaceutical products containing loratadine as the active ingredient are commercially available in the market as CLARITIN™. The tablets contain 10 mg of micronized loratadine, to be administered orally.

U.S. Pat. No. 4,282,233 discloses loratadine, its related compounds, and compositions containing them. It also discloses a process for the preparation of loratadine in which loratadine is finally recrystallized from isopropyl ether after decolorization with decolorizing carbon.

U.S. Pat. No. 6,335,347 discloses polymorphic Form II of loratadine and a process for its preparation. It designates the polymorphic obtained in U.S. Pat. No. 4,282,233 as polymorphic Form I.

The process of the present invention has advantages of improved yield and increased productivity which affords a significantly greater amount of polymorphic Form I of loratadine. The process is also industrially scaleable, and cost effective.

### SUMMARY OF THE INVENTION

The present invention relates to a process for the preparation of polymorphic Form I of loratadine. In particular, the present invention relates to a process for the preparation of polymorphic Form I of loratadine substantially free of polymorphic Form II.

One aspect of the present invention relates to a process for the preparation of polymorphic Form I of loratadine substantially free of polymorphic Form II. In an embodiment, the process comprises:

a) providing a solution of loratadine in a nitrile solvent or a hydrocarbon solvent; and

b) crystallizing a solid from the solution.

An embodiment of the invention provides a process for preparing loratadine Form I, comprising:

a) providing a solution of loratadine in a solvent comprising toluene, xylene, n-heptane, or cyclohexane; and

b) crystallizing a solid from the solution.

Another embodiment of the invention provides a process for preparing loratadine Form I, comprising:

a) providing a solution of loratadine in a solvent comprising acetonitrile or propionitrile; and

b) crystallizing a solid from the solution.

Another aspect of the present invention provides a pharmaceutical composition comprising polymorphic Form I of loratadine substantially free of polymorph Form II of loratadine, prepared according to the process of the present invention, along with one or more pharmaceutically acceptable excipients.

### BRIEF DESCRIPTION OF THE DRAWINGS

FIG. 1 is an X-ray powder diffraction (“XRPD”) pattern of polymorphic Form I of loratadine.

FIG. 2 is an XRPD pattern of polymorphic Form II of loratadine.

FIG. 3 is an XRPD pattern of Form I of loratadine that contains 0.8 percent w/w of Form II.

FIG. 4 is a quantification curve using XRPD for loratadine Form I substantially free of loratadine Form II, prepared in Example 2.

### DETAILED DESCRIPTION OF THE INVENTION

The present invention relates to a process for the preparation of polymorphic Form I of Loratadine. In an aspect, the present invention relates to a process for the preparation of polymorphic Form I of loratadine substantially free of polymorphic Form II.

One aspect of the present invention relates to a process for the preparation of polymorphic Form I of loratadine substantially free of polymorphic Form II. In an embodiment, the process comprises the steps of:

a) providing a solution of loratadine in a nitrile solvent or a hydrocarbon solvent; and

b) crystallizing a solid from the solution.

Step a) involves providing a solution of loratadine in a nitrile solvent or a hydrocarbon solvent.

The solution of loratadine may be obtained by dissolving loratadine in a nitrile solvent or a hydrocarbon solvent, or such a solution may be obtained directly from a reaction in which loratadine is formed in such a solvent.

When the solution is prepared by dissolving loratadine in a nitrile solvent or a hydrocarbon solvent, any form of loratadine such as any crystalline or amorphous form may be utilized for preparing the solution.

Suitable solvents which can be used for dissolving loratadine include but are not limited to: nitrile solvents such as acetonitrile, propionitrile and the like; hydrocarbons such as toluene, xylene, n-heptane, cyclohexane, and the like; and mixtures thereof in various proportions.

The temperatures for preparation of the solution can range from about 20 to 120° C., depending on the solvent used. Any other temperature is also acceptable as long as the stability of loratadine is not compromised.

The quantity of solvent used for preparing the solution depends on the nature of solvent and the temperature adopted for preparing the mixture. The concentration of loratadine in the solution may generally range from about 0.1 to about 10 g/ml in the solvent. In some embodiments of the invention, concentration of the solution, such as to a point near saturation, can be employed to improve product yield.

The solution obtained in step a) can be optionally treated with activated charcoal to enhance the color of the compound followed by filtration through a medium such as through a flux calcined diatomaceous earth (Hyflow) bed to remove the carbon.

The carbon treatment can be conducted either at the temperatures of the preparation of the mixture, or after cooling the solution to lower temperatures.

Step b) involves crystallizing a solid from the solution of step a).

For crystallization to occur, the solution may be maintained at temperatures lower than the solution formation or concentration temperatures, such as for example below about 10° C. to about 25° C., for a period of time as required for a more complete isolation of the product. The exact cooling temperature and time required for complete isolation can be readily determined by a person skilled in the art and will also depend on parameters such as concentration and temperature of the solution or slurry.

Optionally isolation may be enhanced by methods such as cooling, partial removal of the solvent from the mixture, by adding an anti-solvent to the reaction mixture, or a combination thereof.

Suitably, small amounts of seeding crystals of loratadine polymorph Form I may be added to the reaction mixture. Small amounts usually are about 0.01 to 5 weight %, or about 0.01 to 1 weight %. Seeding crystals may be added before or, where appropriate, after the step of initiating the precipitation.

The method by which the solid material is recovered from the final mixture, with or without cooling below the operating temperature, can be any of techniques such as filtration by gravity or by suction, centrifugation, decantation, and the like. The crystals so isolated frequently will carry a small proportion of occluded mother liquor containing a higher percentage of impurities. If desired, the crystals can be washed with a solvent to wash out the mother liquor.

A wet cake from the recovery may optionally be further dried. Drying can be suitably carried out in a tray dryer, vacuum oven, air oven, fluidized bed drier, spin flash dryer, flash dryer and the like. The drying can be carried out at temperatures of about 35° C. to about 70° C. The drying can be carried out for any time periods to achieve a desired product purity, such as from about 1 to 20 hours, or longer.

Loratadine prepared in accordance with the present invention contains less than about 0.5%, or less than about 0.1%, by weight of individual impurities like the loratadine bromo impurity, and the loratadine dehydro impurity, as characterized by a high performance liquid chromatography (“HPLC”) chromatogram obtained from a mixture comprising the desired compound and one or more of the said impurities.

As used herein, “loratadine bromo impurity” refers to 8-Bromo-11-(N-carboethoxy-4-piperidylidine) 6,11-dihydro-5H-benzo[5,6]cyclohepta-[1,2-b]pyridine represented by Formula Ia; and

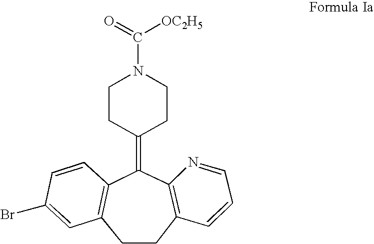

“loratadine dehydro impurity” refers to 8-Chloro-11-(N-Carboethoxy-4-piperidylidine)-5H-benzo[5,6]-cyclohepta-[1,2-b]pyridine represented by Formula Ib.

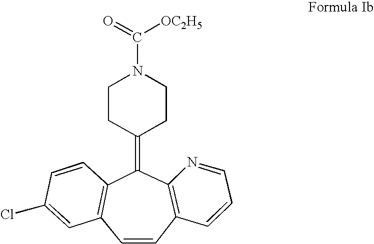

Loratadine Form I obtained according to the process of the present invention is substantially free of Form II.

As used herein “substantially free of Form II” refers to Form I associated with less than about 5 percent by weight Form II, or less than about 2 percent Form II, or less than about 0.8 percent Form II, by weight.

Loratadine Form I prepared in accordance with the present invention is characterized by its XRPD pattern. The XRPD data reported herein were obtained using Cu Kα-1 radiation, having the wavelength 1.541 Å, and patterns were obtained on a Bruker AXS, D8 Advance Powder X-ray Diffractometer.

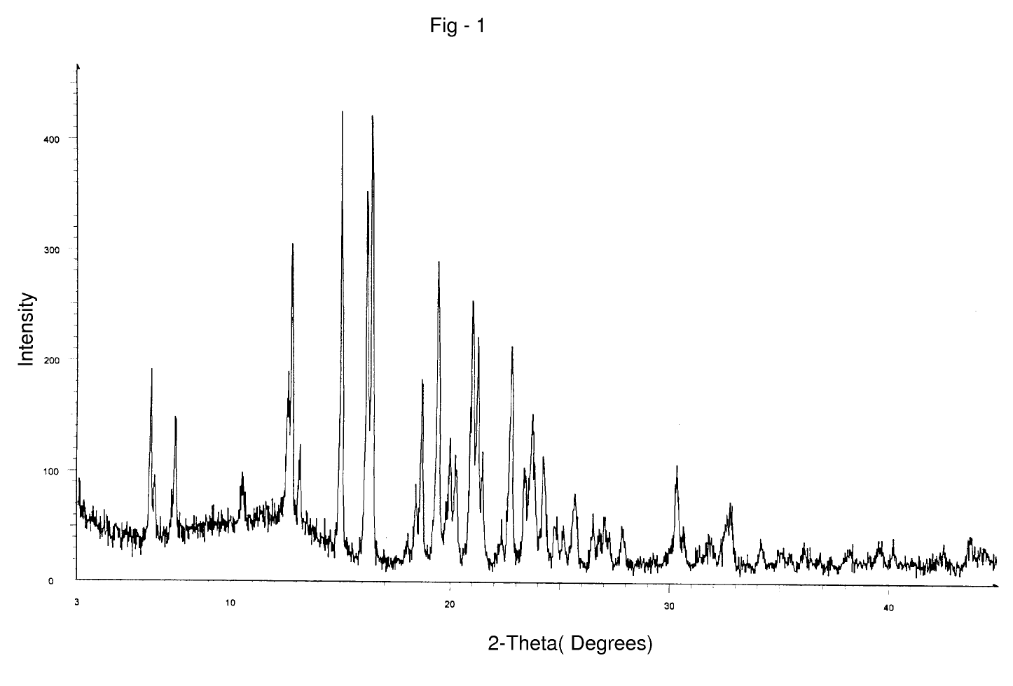

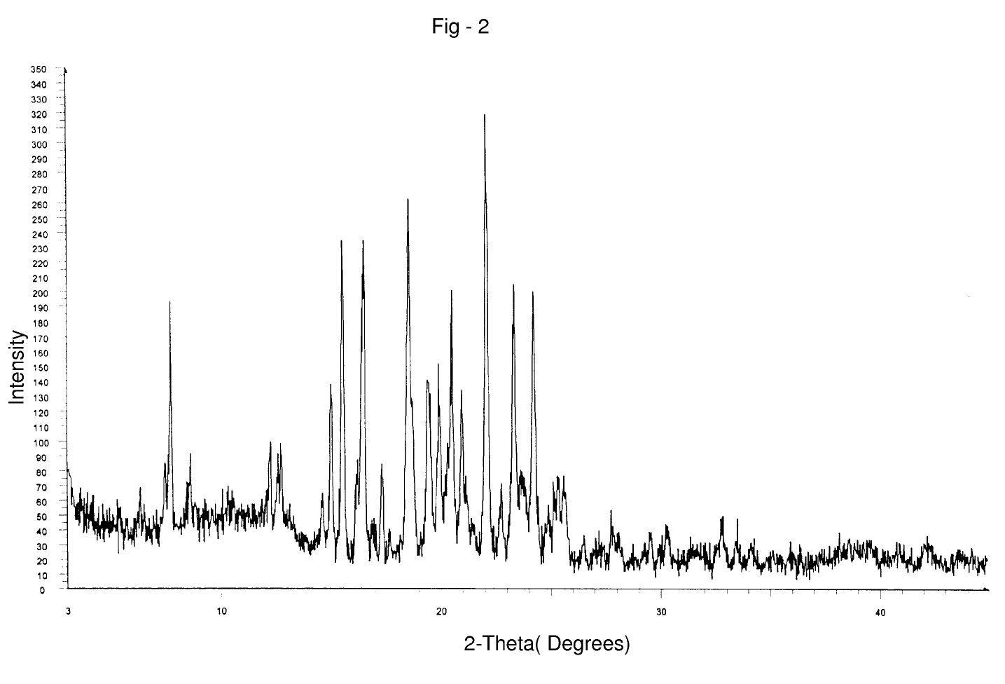

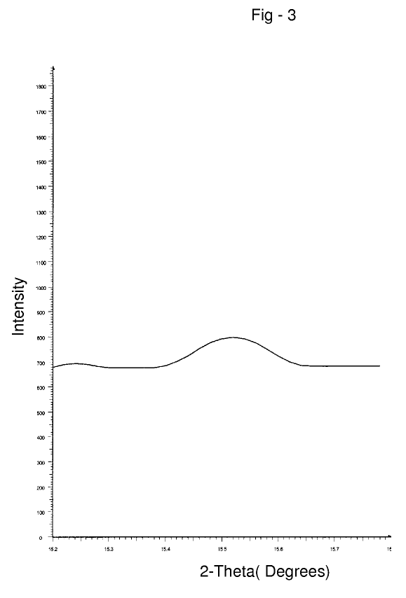

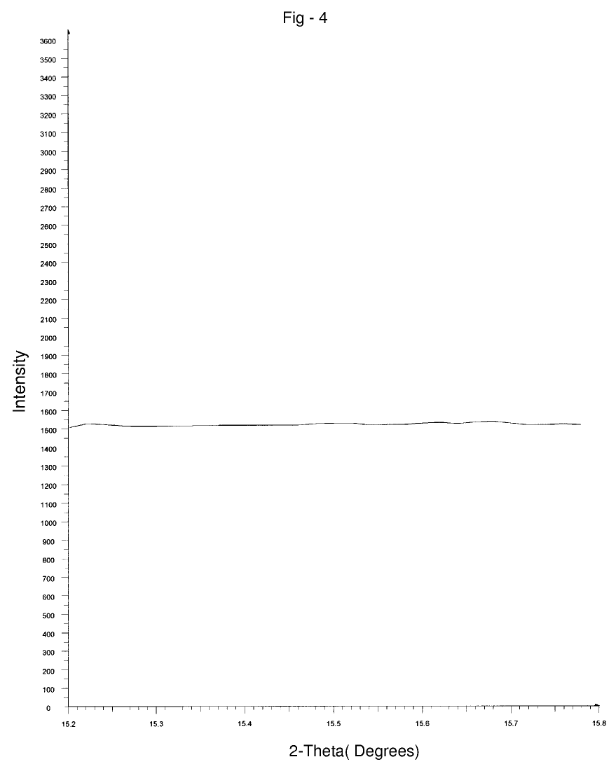

Loratadine Form I and Form II are characterized by their XRPD patterns substantially in accordance with FIGS. 1 and 2, respectively. Form II of loratadine that is contaminating Form I of loratadine can be analyzed using XRPD. The method used herein can detect and quantify as little as 0.8 weight percent of Form II in Form I. The quantification curve for loratadine Form II in Form I of this invention is given in FIG. 3, and the quantification curve for pure Form I is FIG. 4.

The D₁₀, D₅₀, and D₉₀ values are useful ways for indicating a particle size distribution. D₉₀ refers to the value for the particle size for which at least 90 volume percent of the particles have a size smaller than the value. Likewise D₅₀ and D₁₀ refer to the values for the particle size for which 50 volume percent, and 10 volume percent, of the particles have a size smaller than the value. Methods for determining D₁₀, D₅₀, and D₉₀ include laser light diffraction, such as using equipment sold by Malvern Instruments Ltd. of Malvern, Worcestershire, United Kingdom.

Loratadine Form I obtained according to the present invention has: a mean particle size of less than about 50 μm; D₁₀ less than about 10 μm, or less than about 5 μm; D₅₀ less than about 50 μm or less than about 25 μm; and D₉₀ less than about 200 μm, or less than about 100 μm. A Malvern instrument calculates the mean particle size and gives it as D(4,3). It is the average particle size of the powder. There is no specific lower limit for any of the D values.

Loratadine Form I obtained according to the process described in this invention has a bulk density less than about 0.8 g/ml, or less than about 0.5 g/ml, before tapping, and a bulk density less than about 1 g/ml, or less than about 0.5 g/ml, after tapping. The bulk densities are determined using Test 616 “Bulk Density and Tapped Density,” United States Pharmacopeia 24, pages 1913-4 (United States Pharmacopeial Convention, Inc., Rockville, Md., 1999).

Loratadine Form I obtained according to the process of the present invention has a surface area of about 1 to about 2, or from about 0.5 to about 0.9, m²/g.

Specific particle surface area was measured by gas adsorption using a micrometrics GEMINI V series adsorption analyzer, sold by Micromeritics Instrument Corporation, Norcross, Ga. U.S.A. The specific surface area of samples of loratidine particles was calculated by the multipoint Brunauer, Emmet and Teller (BET) method. 0.5 to 1.0 g of pure samples were weighed and thoroughly degassed to remove the adsorbed gases at 40° C. for 1 hour under nitrogen flow before the analysis. Sample tube was evacuated at 200 mm Hg/min pressure for 5 minutes. Free space was measured using helium gas. Adsorption data was generated at −77° C. using N₂ gas (P/P0=0.05 to 0.3) as adsorbate. The instrument software Gemini Confirm V1.00 calculated the moles of adsorbed N₂ for 7 equilibrium pressures. In this range, linearity of the BET equation did not deviate significantly (r=0.999).

Loratadine that is suitable for preparation of polymorphic Form I using the above-described process may be prepared according to processes described in the art, or using a process comprising the steps of:

a) reacting ethylchloroformate with 8-chloro-11-(N-methyl-4-piperidylidene)-6,11-dihydro-5H-benzo-[5,6]-cyclohepta-[1,2-b]-pyridine in the presence of triethylamine in anhydrous toluene; and

b) removing the solvent to get loratadine.

Step a) involves the reaction of ethylchloroformate with 8-chloro-11-(N-methyl-4-piperidylidene)-6,11-dihydro-5H-benzo-[5,6]-cyclohepta-[1,2-b]-pyridine in the presence of triethylamine in anhydrous toluene.

Suitable temperatures for conducting the reaction range form about 20° C. to about 100° C., or from about 50° C. to about 80° C.

After completion of the reaction, the pH of the reaction mass is made alkaline using a suitable base.

Suitable bases which can be used include, but are not limited to: alkali metal hydrides such as lithium hydride, sodium hydride and the like; alkali metal hydroxides such as lithium hydroxide, sodium hydroxide, potassium hydroxide and the like; carbonates of alkali metals such as sodium carbonate, potassium carbonate and the like; bicarbonates of alkali metals such as sodium bicarbonate, potassium bicarbonate, and the like; ammonia; and mixtures thereof. These bases can be used in the form of solids or in the form of aqueous solutions.

Suitably, aqueous solutions containing about 5% to 50%, or about 10% to 20%, (w/v) of the corresponding base can be used. Any concentration is useful, which will convert the acid addition salt to a free base.

The pH is adjusted to from about 7 to about 13 or from about 9 to about 10, which releases the free base of loratadine.

Suitable solvents which can be used for extracting the product include, but are not limited to: esters such as ethyl acetate, n-propyl acetate, n-butyl acetate, t-butyl acetate, and the like; nitriles such as acetonitrile, priopionitrile, and the like; halogenated hydrocarbons such as dichloromethane, ethylene dichloride, chloroform, and the like; hydrocarbon solvents such as toluene, xylene and the like; and mixtures thereof and their combinations with water in various proportions.

Step b) involves removing the solvent to get loratadine.

Suitable techniques which can be used for removal of the solvent include, but are not limited to distillation using a rotational evaporator device such as a Buchi Rotavapor, spray drying, agitated thin film drying (“ATFD”), suppressed boiling type evaporation, and others which use flash evaporation techniques.

Distillation of the solvent may be conducted under a vacuum, such as below about 100 mm Hg to below about 600 mm Hg, at elevated temperatures such as about 20° C. to about 70° C. Any temperature and vacuum conditions can be used as long as there is no increase in the impurity levels of the product.

An embodiment of the invention involves the removal of the solvent using an agitated thin film drying-vertical (“ATFD-V”) technique.

The ATFD-V technique uses high vacuum along with elevated temperatures, which allows operation at lower temperatures. This allows for a short residence time for the product in the drier, which is an important feature for heat sensitive products. The solvent evaporation can be achieved in a single pass, avoiding product recirculation and possible degradation. It is suitable for viscous products, and the operating pressures are from atmospheric down to 1 mbar. The equipment can be operated at a wide range of temperatures, such as 25 to 350° C., or higher.

The concentration, solvent type, temperature, vacuum, and feeding rate are set to conditions, where the loratadine coming from the inlet precipitates instantly without being exposed to elevated temperatures for extended periods of time.

The present process is carried out at lower temperatures of about 35° C. to about 50° C. under reduced pressure of about 600 to about 700 mm Hg. ATFD-V dryers are indirectly heated and therefore air does not come in contact with the product.

The solution of loratadine may be added drop-wise or continuously to the drying chamber. The speed of the addition of the solution will depend on the solvent used, the viscosity of the mixture, and the height of the chamber. The rate of flow may range from 45 to 65 liters/hour. These and other parameters are well known to a person skilled in the art of evaporation using ATFD.

The droplets of solution evaporate almost instantaneously in the chamber. The solidification is spontaneous and does not require further action such as stirring. This instant evaporation allows for obtaining a phase change (solidification) before the solution contacts the bottom of an industrial sized chamber when fed from the top.

The yields obtained using this technique are superior to those obtained using other techniques.

Suitably, loratadine is isolated as a solid from the residue obtained after performing ATFD.

Suitable techniques used for isolation include techniques of crystallization, slurring, or trituration in a suitable solvent.

Suitable solvents which can be used for isolation using the above techniques include, but are not limited to hydrocarbon solvents such as toluene, xylene, n-hexane, n-heptane, cyclohexane and the like; and mixtures thereof.

Depending on the technique chosen, the mixture of loratidine with the solvent may be in the form of a solution or a suspension. The quantity of solvent used depends on the solvent and the temperature adopted for dissolution if it is a solution. The concentration of loratidine in the mixture may generally range from about 0.1 to about 1 g/ml in the solvent.

Another aspect of the present invention provides a pharmaceutical composition comprising polymorphic Form I of loratadine prepared according to the process of the present invention, along with one or more pharmaceutically acceptable excipients.

The pharmaceutical composition comprising loratadine or its salts may be further formulated into solid oral dosage forms such as, but not limited to, powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the form of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared by direct blending, dry granulation or wet granulation or by extrusion and spheronization. Compositions may be presented as uncoated, film coated, sugar coated, powder coated, enteric coated or modified release coated. Compositions of the present invention may further comprise one or more pharmaceutically acceptable excipients.

Pharmaceutically acceptable excipients that find use in the present invention include, but are not limited to: diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, polyvinyl pyrrolidone, hydroxypropyl cellulose, hydroxypropyl methylcellulose, pregelatinized starch and the like; disintegrants such as starch, sodium starch glycolate, pregelatinized starch, crospovidone, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrins, resins; release rate controlling agents such as hydroxypropyl cellulose, hydroxymethyl cellulose, hydroxypropyl methylcellulose, ethyl cellulose, methyl cellulose, various grades of methyl methacrylates, waxes and the like. Other pharmaceutically acceptable excipients that are of use include but are not limited to film formers, plasticizers, colorants, flavoring agents, sweeteners, viscosity enhancers, preservatives, antioxidants and the like.

In the compositions of the present invention, loratadine is a useful active ingredient in the range of about 1 to about 50 mg, per dosage unit.

Certain specific aspects and embodiments of this invention are described in further detail by the examples below, which examples are provided only for the purpose of illustration and are not intended to limit the scope of the appended claims in any manner.

### EXAMPLE 1

### Preparation of Loratadine Polymorphic Form I Substantially Free of Polymorphic Form II

27 Kg of 8-chloro-6,11-dihydro-11-(1-methyl-4-piperidinylidene)-5H-benzo-[5,6]-cyclohepta-[1,2-b]-pyridine was taken into a reactor containing 365 liters of toluene and stirred for about 15 minutes. 41 liters of triethylamine was added to the reactor and the contents were heated to 72.5° C. A mixture of 79 liters of ethylchloroformate and 41 liters of toluene was added to the reaction mass at 70 to 75° C. The reaction mass was stirred for 1 hours 30 minutes at 72-74° C. Reaction completion was confirmed by thin layer chromatography. After the reaction was completed, the reaction mass was cooled to 33.5° C. and 205 liters of water was added slowly. The pH of the reaction mixture was adjusted to 9.3 with 14 liters of 50% aqueous sodium hydroxide solution and stirred for 20 minutes. The aqueous layer was separated and extracted with 135 liters of toluene. The combined organic layer was washed with 410 liters of water in two equal lots. The organic layer was heated to 72° C. and distilled off under a vacuum of 650 mm Hg until 145 liters of the organic layer was left in the reactor. The organic layer was cooled to 40° C. and the solvent was removed completely by using an agitated thin film dryer (ATFD) with an Inlet temperature of 70° C., outlet temperature of 65.7° C., a feed rate of 20-30 L per hour, and a vacuum of 710 mm Hg to get 31.5 Kg of crude loratadine.

The above crude loratadine was charged into a reactor containing 225 liters of cyclohexane and the contents were heated to 76° C. The reaction mass was stirred for 1 hour to get a clear solution. 1.4 Kg of activated charcoal was added into the reaction mass and stirred for 70 minutes. The reaction mass was filtered hot through a leaf filter, a candy filter and a micro filter to remove the charcoal. The carbon bed was washed with 25 liters of cyclohexane. The filtrate was cooled to 48° C. and stirred for 60 minutes. The filtrate was further cooled to 11° C. and stirred for 2.5 hours. The reaction mass was centrifuged and the solid cake was washed with 90 liters of cold cyclohexane in three equal lots. The solid was dried in a cone dryer at 60° C. under a vacuum of 680 mm Hg for 2 hours, 45 minutes to get 25 Kg of the title compound.

### EXAMPLE 2

### Purification of Loratadine Polymorphic Form I Substantially Free of Polymorphic Form II

25 Kg of loratadine Form I obtained using a process described in Example 1 was taken into a reactor containing 327 liters of cyclohexane and the contents were heated to 62° C. The reaction mass was stirred for 40 minutes at 62° C. to get a clear solution. A mixture of 2.5 Kg of activated charcoal and 24 liters of cyclohexane was charged into the reaction mass and stirred for 30 minutes. The reaction mass was filtered hot through a candy filter and a micro filter to remove the charcoal. The reactor was washed with 49 liters of cyclohexane and the washings were filtered hot through a candy filter and a micro filter. The filtrate was cooled slowly to 44° C. and 5 g of loratadine Form I was charged as a seed material. The reaction mass was stirred for 2.5 hours at 40 to 45° C. The mass was further cooled to 11° C. and stirred for 1 hour. The mass was centrifuged and the solid cake was washed with 50 liters of cold cyclohexane in two equal lots. The solid was dried in a cone dryer at 49° C. under a vacuum of about 650 mm Hg for 3 hours, 20 minutes until loss on drying was less than 0.5% w/w, and sieved to yield 20.9 Kg of pure loratadine polymorphic Form I.

### EXAMPLE 3

### Preparation of Loratadine Polymorphic Form I Substantially Free of Polymorphic Form II Using Acetonitrile as the Solvent

1000 ml of toluene was taken into a round bottom flask and 100 g of 8-Chloro-11-(1-methyl-4-piperidine)-6,11-dihydro-5H-benzo[5,6]-cyclohepta-[1,2-b]pyridine was added. 38 g of triethylamine was added to the reaction mixture and the reaction mass was heated to 65° C. 143 ml of ethyl chloroformate was then added to the reaction mass through a pressure equalizing funnel at 69 to 75° C. The temperature of the reaction mass was then increased to 77° C. and maintained for 2 hours and 15 minutes. Reaction completion was checked using thin layer chromatography. After the reaction was completed, the toluene was distilled at a temperature of 75° C. under a vacuum of below 700 mm Hg. 900 ml of toluene was then added to the residue obtained after distillation. The toluene layer was washed with 500 ml of water followed by washing with 1400 ml of 10% aqueous solution of sodium bicarbonate in two equal lots. Finally, the organic layer was washed with 500 ml of water. The organic layer was distilled completely under a vacuum of 700 mm Hg at 70° C. The residue was then co-distilled with 300 ml of acetonitrile in 3 equal lots at 69° C. 300 ml of acetonitrile was added to the resulting residue and 10 g of carbon was added to it and stirred at 77° C. for 30 minutes. The mass was filtered under a hot condition at 75° C. The filter bed was washed with 100 ml of acetonitrile. As the filtrate cooled, a precipitate formed, so the filtrate was reheated to 78° C. for clear dissolution and then was gradually cooled to 30° C. and maintained for 1 hour. The mass was further cooled to 2° C. and maintained for 2 hours. The separated solid was filtered and washed with 50 ml of chilled acetonitrile. The wet solid was dried at about 60° C. to yield 103 g of the title compound.

### EXAMPLE 4

### Preparation of Loratadine Polymorphic Form I Substantially Free of Polymorphic Form II

1000 ml of toluene was taken into a round bottom flask and 100 g of 8-Chloro-11-(1-methyl-4-piperidine)-6,11-dihydro-5H-benzo[5,6]-cyclohepta-[1,2-b]pyridine was added and the reaction mass was stirred at 30° C. for 15 minutes to get a clear solution. 38 g of triethylamine was added to the reaction mixture, and then the reaction mass was heated to 66° C. 143 ml of ethyl chloroformate was then added to the reaction mass at 66° C. The temperature of the reaction mass was then increased to 78° C. and maintained for 4 hours, 15 minutes. Reaction progress was monitored using thin layer chromatography. After the reaction completed, the toluene was distilled at a temperature of 65° C. under a vacuum below 700 mm Hg. 900 ml of toluene was then added to the residue obtained after distillation. The toluene layer was washed with 500 ml of water followed by washing with 1400 ml of a 10% aqueous solution of sodium bicarbonate in two equal lots. Finally, the organic layer was washed with 500 ml of water. The organic layer was distilled completely under a vacuum of 700 mm Hg at 70° C. The residue was then co-distilled with 300 ml of cyclohexane in 3 equal lots at 69° C. 500 ml of cyclohexane was added to the resulting residue and 10 g of carbon was added and stirred at 76° C. for 20 minutes. The mass was filtered in a hot condition at 75° C. The filter bed was washed with 100 ml of cyclohexane. The reaction mass was gradually cooled to 30° C. and maintained for 1 hour. The reaction mass was further cooled to 5° C. and maintained for 2 hours. The separated solid was filtered and washed with 50 ml of chilled cyclohexane. The wet solid was dried at about 55° C. for 2.5 hours to yield 106 g of the title compound.

### EXAMPLE 5

### Detection of Loratadine Form II in Form I by X-Ray Diffraction

### Standard Preparation:

992 mg of loratadine Form 1 and 8.0 mg of Form 2 were ground together three times to get a uniform powder.

### Test Sample Preparation:

Accurately 1 g of the test sample was weighed and ground 3 to 4 times to get a homogeneous sample.

### Typical Experimental Conditions:

Experimental conditions for performing the XRPD analysis for quantification were as follows:

Instrument used: Powder X-ray diffractometer.

Make, Model: Bruker AXS, D8 Advance.

Goniometer: Theta/theta vertical.

Measuring circle: 435 nm.

Radiation: Cu K alpha-1 (Wavelength=1.5406 Å).

Tube: 2.2 kw Copper long fine focus.

Detector: Scintillation counter.

Voltage, current: 40 kV, 50 mA.

Scan type: Locked couple.

Scan mode: Step scan.

Divergence Slit: 1.0 degree.

Antiscattering Slit: 1.0 degree.

Detector slit: 0.2 nm.

Synchronous rotation: On.

Scan Range: 15.2° to 15.8° 2θ, and ref peak at 5.70° to 6.70°.

Step size 0.02°.

Time/step: 40.0 seconds and ref peak 20.0 seconds.

### EXAMPLE 5

### Determination of Impurities in Loratadine

Determining the level of impurities in loratadine using HPLC. The HPLC analysis conditions are described in Table 1.

TABLE 1
HPLC method for detecting the level of the impurities.

| Apparatus: | A liquid chromatograph equipped with variable | | |
|---|---|---|---|
| | wavelength detector and integrator. | | |
| Column: | Intersil ODS-3V, 250 × 4.6 mm ID, 5.0 μ. | | |
| Buffer: | 6.8 g of potassium dihydrogen phosphate dissolved in | | |
| | 1000 ml of water and pH adjusted to 2.8 with dilute | | |
| | phosphoric acid. | | |
| Mobile Phase A: | Mixed buffer, methanol and acetonitrile in a ratio | | |
| | 350:300:400 v/v. | | |
| Mobile Phase B: | A mixture of acetonitrile and water in a ratio of | | |
| | 800:200 v/v. | | |
| Gradient | Time (minutes) | % A | % B |
| program: | 0.01 | 100 | 0 |
| | 30 | 100 | 0 |
| | 40 | 5 | 95 |
| | 50 | 5 | 95 |
| | 52 | 100 | 0 |
| | 65 | 100 | 0 |

| | |
|---|---|
| Temperature: | 27° C. |
| Injection volume: | 20 μL |
| Flow rate: | 1.2 mL per minute |
| Detector: | 220 nm |
| Diluent: | Mobile phase A |
| Sample | 0.5 mg/mL in diluent |
| concentration: | |
| Run time: | 65 minutes |

| IMPURITY NAME | RRT |
|---|---|
| Loratadine bromo impurity | 1.1 |
| Loratadine dehydro impurity | 1.2 |

The relative retention times (“RRT”) are calculated by dividing actual retention times (“RT”) by the retention time for loratadine.

1. A process for preparing loratadine Form I, comprising:
a) providing a solution of loratadine in a nitrile solvent or a hydrocarbon solvent; and
b) crystallizing a solid from the solution.

2. The process of claim 1, wherein a hydrocarbon solvent comprises toluene, xylene, n-heptane, or cyclohexane.

3. The process of claim 1, wherein a nitrile solvent comprises acetonitrile or propionitrile.

4. The process of claim 1, wherein loratadine is prepared by a process comprising:
a) reacting ethylchloroformate with 8-chloro-11-(N-methyl-4-piperidylidene)-6,11-dihydro-5H-benzo-[5,6]-cyclohepta-[1,2-b]-pyridine in the presence of triethylamine in anhydrous toluene; and
b) removing solvent to recover loratadine.

5. The process of claim 1, wherein loratadine Form I contains less than about 5 weight percent of loratadine Form II.

6. The process of claim 1, wherein loratadine Form I contains less than about 2 weight percent of loratadine Form II.

7. The process of claim 1, wherein loratadine Form I contains less than about 0.8 weight percent of loratadine Form II.

8. The process of claim 1, wherein loratadine Form I contains less than about 0.15 weight percent of a compound having a formula: 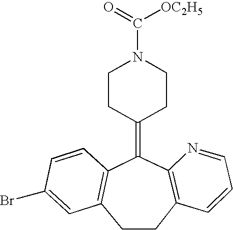
and less than about 0.15 weight percent of a compound having a formula:

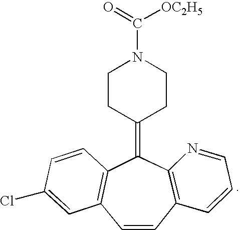

9. The process of claim 1, wherein loratadine Form I contains less than about 0.1 weight percent of a compound having a formula: 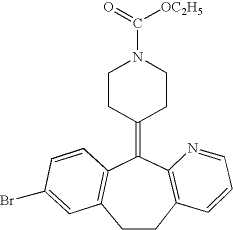
and less than about 0.1 weight percent of a compound having a formula:

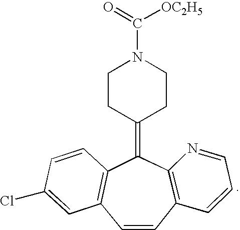

10. The process of claim 1, wherein loratadine Form I has a mean particle size less than about 50 μm, D₁₀ less than about 10 μm, D₅₀ less than about 50 μm, and D₉₀ less than about 200 μm.

11. The process of claim 1, wherein loratadine Form I has a bulk density less than about 0.8 g/ml, before tapping, and a bulk density less than about 1 g/ml, after tapping.

12. The process of claim 1, wherein loratadine Form I has a surface area about 1 to about 2 m²/g.

13. A process for preparing loratadine Form I, comprising:
a) providing a solution of loratadine in a solvent comprising toluene, xylene, n-heptane, or cyclohexane; and
b) crystallizing a solid from the solution.

14. The process of claim 13, wherein loratadine Form I contains less than about 5 weight percent of loratadine Form II.

15. The process of claim 13, wherein loratadine Form I contains less than about 2 weight percent of loratadine Form II.

16. The process of claim 13, wherein loratadine Form I contains less than about 0.8 weight percent of loratadine Form II.

17. A process for preparing loratadine Form I, comprising:
a) providing a solution of loratadine in a solvent comprising acetonitrile or propionitrile; and
b) crystallizing a solid from the solution.

18. The process of claim 17, wherein loratadine Form I contains less than about 5 weight percent of loratadine Form II.

19. The process of claim 17, wherein loratadine Form I contains less than about 2 weight percent of loratadine Form II.

20. The process of claim 17, wherein loratadine Form I contains less than about 0.8 weight percent of loratadine Form II.
